(19) United States
(12) Patent Application Publication
(10) Pub. No.: US 2025/0243519 A1
(43) Pub. Date: Jul. 31, 2025

(54) CONTINUOUS METHOD FOR GROWING A MICROORGANISM

(71) Applicant: SPECIALTY OPERATIONS FRANCE, Lyon (FR)

(72) Inventors: Laurent Garel, Lyon (FR); Guillaume Pireau, Lyon (FR); Chenglu David, Lyon (FR)

(73) Assignee: SPECIALTY OPERATIONS FRANCE, Lyon (FR)

(21) Appl. No.: 18/855,630

(22) PCT Filed: Apr. 14, 2023
(86) PCT No.: PCT/EP2023/059804

§ 371 Date: Oct. 9, 2024

(30) Foreign Application Priority Data
Apr. 15, 2022 (FR) 2203533

(51) Int. Cl. C12P 7/22; C12P 7/26

(52) U.S. Cl. CPC C12P 7/22; C12P 7/26

(57) ABSTRACT

The present invention relates to a method comprising a step (a) of continuously growing a microorganism capable of transforming a substrate into a compound of formula (I), preferably vanillin.

### FIELD OF THE INVENTION

The present invention relates to a process comprising a step (a) of continuously growing a microorganism capable of converting a substrate into a compound of formula (I), preferably vanillin.

### PRIOR ART

Vanillin may be obtained by various methods known to a person skilled in the art, notably by the following two routes:
a “natural” route based on a biotechnological process comprising in particular the culturing of a microorganism capable of enabling the bioconversion of a fermentation substrate into vanillin. Such a process, wherein the fermentation substrate is ferulic acid, is notably known from patent application EP 0885968. U.S. Pat. No. 5,017,388 describes a process wherein the fermentation substrate is eugenol and/or isoeugenol. These processes result in the preparation of a vanillin known as natural vanillin;
a “synthetic” route comprising conventional chemical reactions starting from guaiacol not involving a microorganism. This process leads to the preparation of a vanillin known as synthetic vanillin.

Finally, vanillin may also be prepared via a “biobased” route, wherein the vanillin is derived from lignin. In particular, mention may be made of U.S. Pat. No. 2,745,796, DE 1132113 and the article entitled “Preparation of lignin from wood dust as vanillin source and comparison of different extraction methods” by Azadbakht et al. in International Journal of Biology and Biotechnology, 2004, vol. 1, No. 4, pages 535-537, or prepared from materials of natural origin. Mention may particularly be made of document WO 2019/020773.

As mentioned above, the processes of the “natural” route comprise culturing a microorganism capable of converting a substrate into vanillin. This step is a phase of growing the microorganism to obtain a biomass that will be used as a biocatalyst for the phase of bioconversion of a substrate into vanillin.

Industrially, this growth phase is time-consuming and tricky. Indeed, it is generally necessary to inoculate a small amount of microorganism contained in a cryotube, and to transfer it into a first fermentor of slightly larger volume in order to grow the microorganism. This step of growing the biomass and transferring into a larger reactor may be repeated as many times as necessary. The total duration of this growth step may be considerable, of the order of 72 h for example.

Moreover, this growth step must be carried out in a sterile medium. An initial step of sterilizing the fermentor(s) and all inlets and outlets of the fermentor(s) is required and must be carried out under specific conditions. As a result, on the one hand the transfers from one fermentor to another present risks of contamination and, on the other hand, the implementation of this type of process is complex.

The present invention is directed toward an efficient and industrial process for growing a microorganism, wherein the risk of contamination is reduced and the productivity is improved.

### BRIEF DESCRIPTION

The present invention firstly relates to a process comprising a step (a) of continuously growing a microorganism capable of converting a substrate into a compound of formula (I), preferably vanillin.

The present invention also relates to a process for producing a compound of formula (I), preferably vanillin, characterized in that it comprises a step (a) of continuously growing a microorganism capable of converting a substrate into a compound of formula (I), preferably vanillin.

### DESCRIPTION OF THE FIGURES

FIG. 1: Continuous growth process according to the invention

FIG. 2: Process for producing a compound of formula (I) according to the invention

FIG. 3: Process for producing a compound of formula (I) according to the invention

FIG. 4: Process for producing a compound of formula (I) according to the invention

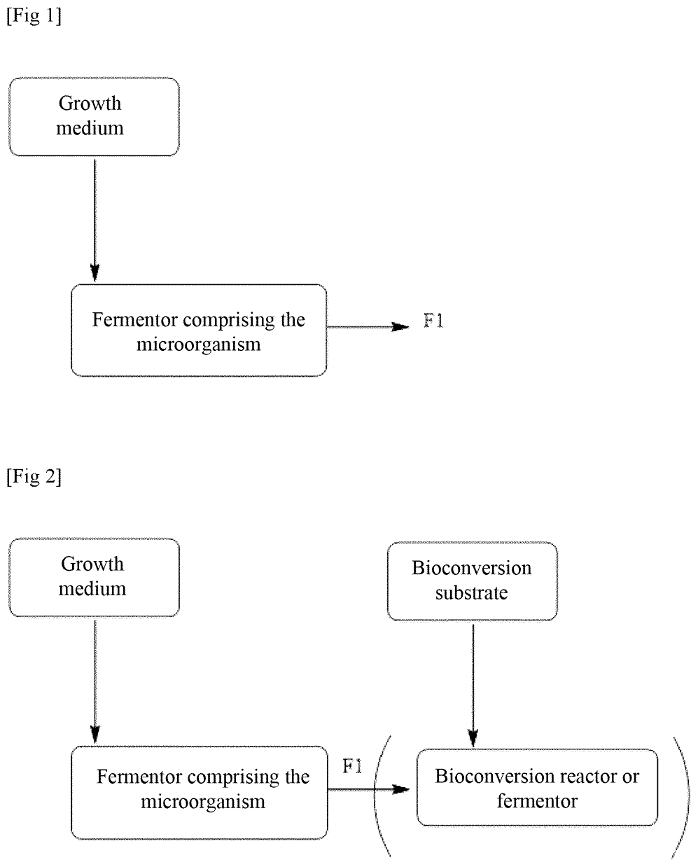

### DETAILED DESCRIPTION

In the context of the present invention, and unless otherwise indicated, the expression “between . . . and . . . ” includes the limits.

In the context of the present invention, and unless otherwise indicated, the growth of a microorganism refers to a process wherein said microorganism multiplies. The growth leads to the production of biomass. The growth may be defined by the increase in the concentration of the biomass.

In the context of the present invention, and unless otherwise indicated, the term “bioconversion” refers to a biotechnological process wherein a microorganism allows the conversion of a substrate into a bioconversion product. The microorganism may be a genetically modified wild-type strain that has been obtained by molecular biology or mutated, in particular by random or site-directed mutagenesis.

In the context of the present invention, and unless otherwise indicated, the term “process carried out under aseptic conditions” refers to a process carried out under conditions free of any microbe and/or microorganism.

In the context of the present invention, and unless otherwise indicated, the term “process carried out under sterile conditions” refers to a process carried out under conditions free of any microbe and/or microorganism liable to be detrimental to the microorganism capable of converting a substrate into a compound of formula (I), preferably vanillin, used in the context of the process of the present invention.

In the context of the present invention, and unless otherwise indicated, the asepsis or sterilization may be obtained by any method, in particular thermal, chemical, mechanical or radiation method, enabling the elimination of microbes and/or microorganisms. The asepsis or the sterilization may be performed on the fluids, starting materials, and/or devices used in the context of the process according to the present invention. By way of illustration, mention may be made in particular of the following methods:
Wet- or dry-heat sterilization, carried out by raising the temperature to a sufficient temperature, followed by maintaining this temperature for a sufficient period of time to eliminate the microbes and/or microorganisms; by way of illustration, the temperature may be 121° C., followed by the maintaining of this temperature of 121° C. for a sufficient period of time to eliminate the microbes,
Membrane filtration or several successive membrane filtrations, the smallest cut-off diameter of which allows the removal of any microbe and/or microorganism.

In the context of the present invention, and unless otherwise indicated, the term “reactor” refers to a vessel suitable for carrying out chemical reactions. In the context of the present invention, the reactor may be used after having been made aseptic or sterilized or without aseptization or sterilization.

In the context of the present invention, and unless otherwise indicated, the term “fermentation” refers to any biological reaction involving a microorganism, such as bacterial growth, bioconversion, biosynthesis or biocatalysis.

In the context of the present invention, and unless otherwise indicated, the term “fermentor” refers to a reactor suitable for carrying out fermentations, for example a bioreactor. In the context of the present invention, the fermentor may be used after having been made aseptic or sterilized or without aseptization or sterilization.

In the context of the present invention, a compound of formula (I) has the following formula:

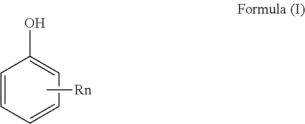

wherein n is between 0 and 5, preferably between 1 and 2,
R, which may be identical or different, is chosen from the group consisting of OH, OR₁, formyl, CO₂H, optionally substituted linear or branched alkyl chains comprising between 1 and 6 carbon atoms, optionally substituted linear or branched alkenyl chains comprising between 1 and 6 carbon atoms.

According to a particular embodiment, R is chosen from the group consisting of formyl —CHO, OH, OMe, OEt, OPr, OBu, methyl, ethyl, —(CH₂)₂—C(O)—CH₃, —CH═CH—C(O)—CH₃, —CH═CH—CO₂H, —CO₂H, —CH₂—OH.

According to a particular embodiment, R₁ is chosen from the group consisting of methyl, ethyl, propyl and butyl.

According to a particular embodiment, the compound of formula (I) is chosen from vanillin and frambinone.

A first aspect of the present invention relates to a process comprising a step (a) of continuously growing a microorganism capable of converting a substrate into a compound of formula (I), preferably vanillin.

In the context of the present invention, the process comprising a step (a) of continuously growing or “continuous growth process” refers to a step or a process wherein the volume of the reactor is constant. During the continuous growth process, the microorganism multiplies. During the continuous growth process, a stream (F1) comprising at least the biomass is continuously transferred. According to one embodiment, a stream (F1) comprising the entire biomass is continuously transferred. According to another embodiment, a stream (F1) comprising a portion of the biomass is continuously transferred.

In the context of the present invention, the pH of the growth medium is controlled and optionally regulated by addition of acid or base. A person skilled in the art will be able to adjust the pH of the growth medium to the needs of the microorganism capable of converting a substrate into a compound of formula (I), preferably into vanillin, used in the context of the process of the present invention.

According to a particular embodiment, the pH of the growth medium is between 3 and 9, in particular between 4 and 8, and very preferentially between 6 and 8.

In the context of the present invention, the continuous growth process is carried out under controlled temperature conditions, it being possible for the temperature to be optionally be regulated. A person skilled in the art will be able to adjust the temperature of the process to the needs of the microorganism capable of converting a substrate into a compound of formula (I), preferably into vanillin, used in the context of the process of the present invention. According to a particular embodiment, the temperature may be between 1° and 55° C., particularly preferably within the range of 30 to 50° C., and very preferentially between 35° C. and 45° C.

In the context of the present invention, the pO₂ of the growth medium is controlled and optionally regulated. A person skilled in the art will be able to adjust the pO₂ of the growth medium to the needs of the microorganism capable of converting a substrate into a compound of formula (I), preferably into vanillin, used in the context of the process of the present invention. By way of illustration, the pO₂ may be between 1% and 99%, preferably of the order of 10%, 20%, 30%, 40%, 50%, 60%, 70%, 80%, 90%. The pO₂ corresponds to the dissolved oxygen concentration in the growth medium relative to the dissolved oxygen concentration at saturation, without biomass. The pO₂ may in particular be measured with a Hamilton Oxyferm FDA VP 225 O₂ Sensor.

In the context of the present invention, the continuous growth process is carried out with stirring. The stirring may be characterized by virtue of the blade tip speed or peripheral speed. The blade tip speed may be calculated with the following formula:

VP=2*Pi*D/2*N/60, wherein Vp is the blade tip speed (m/s), D is the diameter of the reactor or fermentor (in m) and N is the speed of rotation of the stirrer (rpm). A person skilled in the art will be able to adjust the stirring to the needs of the microorganism capable of converting a substrate into a compound of formula (I), preferably into vanillin, used in the context of the process of the present invention. According to a particular embodiment, the blade tip speed is between 1 and 10 m/s, preferably between 2 and 5 m/s.

According to the present invention, the growth is carried out in a medium or “growth medium” suitable for the microorganism for which the growth is sought. In general, the growth medium refers to a medium comprising nutrients essential and/or beneficial to the survival and/or growth of the microorganisms.

In the context of the present invention, the composition of the growth medium is controlled. The composition of the growth medium is continuously adjusted. Thus, in the context of the present invention, growth medium is added continuously.

In the context of the present invention, the rate of addition of the growth medium is equal to the rate of withdrawal of the stream (F1). In the context of the present invention, it is preferable for the rate of addition of the growth medium or of withdrawal of the stream to be adjusted so as to allow the microorganism sufficient time to multiply and to avoid a decrease in its concentration. It is preferable for the rate of addition of the growth medium or of withdrawal of the stream to be adjusted so as to avoid accumulation or reduction of the amount of glucose in the medium.

According to one embodiment, the rates of addition of the growth medium and of withdrawal of the stream (F1) may be constant. According to another embodiment, the rates of addition of the growth medium and of withdrawal of the stream (F1) may be intermittent.

In general, the continuous growth process according to the present invention is carried out in a fermentor. In general, the continuous growth process is carried out under aseptic conditions, preferably under sterile conditions.

In the context of the present invention, the expression “the continuous growth process is carried out under aseptic conditions, preferably sterile conditions” indicates that the reactor or fermentor in which the process is carried out, and the various feed or outlet pipes of the reactor or fermentor, have been rendered aseptic, preferably sterilized, prior to their use in the process. In the context of the present invention, the expression “the continuous growth process is carried out under aseptic conditions, preferably sterile conditions” indicates that the solvent and the growth medium used during the process have been rendered aseptic, preferably sterilized, prior to their use in the process. In the context of the present invention, the expression “the continuous growth process is carried out under aseptic conditions, preferably sterile conditions” indicates that the air or the oxygen used during the process has been rendered aseptic, preferably sterilized, prior to its use in the process.

In general, the growth medium includes a carbon source, an organic or inorganic nitrogen source, inorganic salts and, optionally, growth factors. A person skilled in the art will be able to adjust the composition of the growth medium to the needs of the microorganism capable of converting a substrate into a compound of formula (I), preferably into vanillin, used in the context of the process of the present invention.

In general, the term “carbon source” refers to a carbon source that could be used to support the growth of microorganisms. Cells usually use sugars as their primary carbon source and energy source. However, other carbon sources may also be used. According to a particular aspect, the carbon source may be chosen from the group consisting of glucose, fructose, sucrose, mannose, xylose, arabinose, galactose, lactose, ethanol, cellobiose, glycerol and polysaccharides such as cellulose, and mixtures thereof. According to a preferred embodiment, the carbon source comprises glucose.

In the context of the present invention, the concentration of carbon source is controlled. In the context of the present invention, the concentration of carbon source is adjusted to the needs of the microorganism capable of converting a substrate into a compound of formula (I), preferably into vanillin, used in the context of the process of the present invention.

In the context of the present invention, the concentration of carbon source is maintained at a concentration of between 0 and 20 g·l⁻¹, preferably between 0.1 and 10 g·l⁻¹ and very preferentially between 0.5 and 5 g·l⁻¹. The addition of a carbon source, in particular glucose, is generally carried out continuously.

In general, the term “nitrogen source” refers to a nitrogen source that could be used to support the growth of microorganisms. Examples of suitable nitrogen sources are inorganic nitrogen sources, such as nitrates and ammonium salts, and organic nitrogen sources such as yeast extract, urea or peptone.

The inorganic salts which may be used are, for example, inter alia, sulfates, nitrates, chlorides, carbonates, and hydrogen phosphates and phosphates of sodium, potassium, magnesium, calcium, zinc and iron.

In the context of the present invention, growth factors that may be used are, for example, D-biotin, nicotinic acid, thiamine (HCl), pyridoxine (HCl), para-aminobenzoic acid, myoinositol, calcium pantothenate, Na₂-EDTA, ZnSO₄·7H₂O, MnCl₂·4H₂O, CoCl₂·6H₂O, CuSO₄·5H₂O, Na₂MoO₄·2H₂O, CaCl₂·2H₂O, FeSO₄·7H₂O, H₃BO₃, KI.

In the context of the present invention, the concentration of nitrogen source and/or growth factors may be controlled. In the context of the present invention, the concentration of nitrogen source and/or growth factors may be between 0 and 5 g·l⁻¹, preferably between 0.1 and 2.5 g·l⁻¹, and very preferentially between 0.2 and 1.5 g·l⁻¹.

In addition, magnesium ions, such as magnesium sulfate, may be added at a concentration of between 0 and 0.5 g·l⁻¹, preferably between 0.01 and 0.25 g·l⁻¹, and very preferentially between 0.02 and 0.15 g·l⁻¹. The addition of magnesium ions may be continuous.

In the context of the present invention, the biomass concentration may be controlled. The biomass concentration, expressed as dry mass, may be between 2 and 50 g/l, preferably between 5 and 25 g/l, very preferentially between 8 and 15 g/l.

In the context of the present invention, the biomass concentration may be controlled by measuring the optical density. In general, the optical density, measured at 880 nm, is between 5 and 400, preferably between 20 and 200, very preferentially between 40 and 100 and even more preferentially between 60 and 80. The measurement of the optical density at 880 nm may be performed continuously, online. The optical density at 880 nm may be measured using a Hamilton Dencytec Unit probe.

In the context of the present invention, the biomass concentration may be controlled by measuring the optical density at 600 nm. In general, the optical density, measured at 600 nm, is between 2 and 100, preferably between 5 and 80, very preferentially between 10 and 60 and even more preferentially between 25 and 45. The measurement of the optical density at 600 nm may be performed by taking a sample of growth medium. The sample is then diluted 50 times in water. The optical density at 600 nm may be measured with a spectrophotometer set to 600 nm.

In the context of the present invention, the microorganism may be any microorganism capable of forming a compound of formula (I), preferably vanillin, by bioconversion of an appropriate substrate. The microorganism, in particular bacterium or fungus, may be a wild-type microorganism that has been genetically modified by molecular biology, or mutated, by random or site-directed mutagenesis. Preferably, the microorganism is chosen from bacteria belonging to the order Actinomycetales, Caulobacterales or Pseudomonadales, preferably belonging to the family Streptomycetacae, Pseudonocardiacae, Micrococcaceae, Caulobacteraceae or Pseudomonadaceae, very preferentially Streptomyces setonii, Amycolatopsis sp., Streptomyces psammoticus, Amycolatopsis thermoflava, Micrococcus sp., Caulobacter segnis, Pseudomonas fluorescens, or mutants thereof, even more preferentially Streptomyces setonii, Amycolatopsis sp., Streptomyces psammoticus. Preferably, the bioconversion reaction is carried out in the presence of a strain available under the number ATCC39116, DSMZ 9991, DSMZ 9992, CCTCC 2015329, CCTCC 2011265, IMI 390106 or Zyl 926 or mutants thereof, preferentially ATCC39116, DSMZ 9991, DSMZ 9992, CCTCC 2015329, IMI 390106 or Zyl 926 or mutants thereof and very preferentially ATCC39116, DSMZ 9991, DSMZ 9992, CCTCC 2015329, IMI 390106 or Zyl 926. According to another embodiment, the microorganism may be chosen from genetically modified microorganisms belonging to the family Saccharomyces cerevisiae, Schizosaccharomyces pombe, E. coli, Corynebacterium glutamicul.

The microorganism used in the context of the present invention may also be a strain as described in WO 2014/102368, WO 2016/001203 or EP 2721148.

According to another embodiment, the microorganism is chosen from fungi belonging to the genus Aspergillus, Pycnoporus, Penicillium, preferably belonging to the family Aspergillus luchuensis, Aspergillus niger, Pycnoporus cinnabarinus, Penicillium camamberti.

In the context of the present invention, the substrate may be converted by bioconversion into a compound of formula (I), preferably into vanillin. The substrate is generally chosen from the group consisting of glucose, ferulic acid, eugenol, isoeugenol, coumaric acid, caffeic acid, L-tyrosine, vanillic acid and 4-vinyl guaiacol, preferably chosen from glucose, ferulic acid and eugenol.

In general, the continuous growth process may be preceded by a step (a0) during which a microorganism is cultured so as to obtain a sufficient amount of biomass. This sufficient amount of biomass may then be used in a continuous growth process according to the present invention. The culturing of the bacterium is generally performed in an aqueous medium, in the presence of nutrient elements. In general, the culture medium comprises a carbon source, an organic or inorganic nitrogen source, inorganic salts and growth factors. A person skilled in the art will be able to adjust the composition of the culture medium to the needs of the microorganism capable of converting a substrate into a compound of formula (I), preferably into vanillin, used in the context of the process of the present invention. The concentration of the carbon source is generally between 5 and 50 g·l⁻¹, preferably between 20 and 34 g·l⁻¹. The nitrogen source, such as a yeast extract, and the growth factors are generally added at a concentration of between 2 and 20 g·l⁻¹, preferably between 5 and 10 g·l⁻¹. In addition, magnesium ions, such as magnesium sulfate, may be added at a concentration of between 0.1 and 5 g·l⁻¹, preferably between 0.5 and 1 g·l⁻¹. In general, the pH of the culturing is between 3 and 9, in particular between 4 and 8, and very preferentially between 6 and 8. In general, the temperature of the culturing is between 1° and 55° C., particularly preferably within the range of 30 to 50° C., preferentially between 3° and 42° C. and very preferentially between 35° C. and 45° C.

The culture period generally lasts between 15 minutes and 80 hours, preferably between 1 hour and 50 hours and very preferentially between 5 hours and 40 hours. The duration of the culture period is variable and may be adjusted as a function of the microorganism capable of converting a substrate into a compound of formula (I), preferably into vanillin, used in the context of the present invention. By way of illustration, the culture period may last until the carbon source, generally glucose, is almost entirely consumed, preferably such that the concentration of carbon source is less than or equal to 15 g·l⁻¹, preferably less than or equal to 5 g·l⁻¹, very preferentially less than or equal to 3 g·l⁻¹. According to another embodiment, the culture period may last until 10%, 20%, 30%, 40%, 50%, 60%, 70%, 80%, 90%, 95% of the carbon source introduced at the beginning of step (a0) has been consumed.

According to another aspect, the present invention relates to a process for producing a compound of formula (I), preferably vanillin, characterized in that it comprises a step (a) of continuously growing a microorganism capable of converting a substrate into a compound of formula (I), preferably into vanillin.

In the context of this embodiment, step (a) is carried out under the conditions described above for the process for continually growing a microorganism capable of converting a substrate into a compound of formula (I), preferably into vanillin.

The process for producing a compound of formula (I), preferably vanillin, may also comprise a step (a0) prior to step (a) as described above.

According to one embodiment, the process for producing a compound of formula (I), preferably vanillin, also comprises a bioconversion step (b) wherein the microorganism converts a substrate into a compound of formula (I), preferably into vanillin.

Steps (a) and (b) may be carried out successively; step (a) is carried out before step (b).

According to another embodiment, steps (a) and (b) may be carried out simultaneously.

The bioconversion step (b) may in particular be carried out under the conditions described in EP0885968, EP0761817 or WO2017/025339.

According to an alternative, step (b) may be carried out batchwise or in fed-batch mode. The bioconversion substrate may be added in one step or in several steps. According to another embodiment, step (b) may be carried out continuously. In particular, bioconversion substrate may be fed continuously. Optionally, a stream (F2) comprising the compound of formula (I), preferably vanillin, may be continuously removed. The stream (F2) may also optionally comprise fermentation substrate, impurities and biomass.

Step (b) may be carried out under aseptic conditions, preferably sterile conditions. According to another embodiment, step (b) may be carried out without aseptic or sterility conditions.

In particular, the bioconversion substrate may be chosen from glucose, ferulic acid, cugenol, isocugenol, coumaric acid, caffeic acid, L-tyrosine, vanillic acid and 4-vinyl guaiacol, preferably chosen from glucose, ferulic acid and eugenol.

In the context of the present invention, the microorganism may be any microorganism capable of forming compound of formula (I), preferably vanillin, by bioconversion of an appropriate substrate. The microorganism, in particular bacterium or fungus, may be a wild-type microorganism that has been genetically modified by molecular biology, or mutated, by random or site-directed mutagenesis. Preferably, the microorganism is chosen from bacteria belonging to the order Actinomycetales, Caulobacterales or Pseudomonadales, preferably belonging to the family Streptomycetacae, Pseudonocardiacae, Micrococcaceae, Caulobacteraceae or Pseudomonadaceae, very preferentially Streptomyces setonii, Amycolatopsis sp., Streptomyces psammoticus, Amycolatopsis thermoflava, Micrococcus sp., Caulobacter segnis, Pseudomonas fluorescens, or mutants thereof, even more preferentially Streptomyces setonii, Amycolatopsis sp., Streptomyces psammoticus. Preferably, the bioconversion reaction is carried out in the presence of a strain available under the number ATCC39116, DSMZ 9991, DSMZ 9992, CCTCC 2015329, CCTCC 2011265, IMI 390106 or Zyl 926 or mutants thereof, preferentially ATCC39116, DSMZ 9991, DSMZ 9992, CCTCC 2015329, IMI 390106 or Zyl 926 or mutants thereof and very preferentially ATCC39116, DSMZ 9991, DSMZ 9992, CCTCC 2015329, IMI 390106 or Zyl 926.

According to another embodiment, the microorganism may be chosen from genetically modified microorganisms belonging to the family Saccharomyces cerevisiae, Schizosaccharomyces pombe, E. coli, Corynebacterium glutamicul.

The microorganism used in the context of the present invention may also be a strain as described in WO 2014/102368, WO 2016/001203 or EP 2721148.

According to another embodiment, the microorganism is chosen from fungi belonging to the genus Aspergillus, Pycnoporus, Penicillium, preferably belonging to the family Aspergillus luchuensis, Aspergillus niger, Pycnoporus cinnabarinus, Penicillium camamberti.

According to a first aspect of the present invention, step (b) is carried out in a batchwise process according to FIG. 2. According to this embodiment, step (a) is carried out in a fermentor. A stream (F1) is drawn from this fermentor to feed a reactor or a fermentor. The bioconversion is carried out under batchwise conditions as described in EP0885968, with or without sterilization of the reactor or fermentor used in the context of the bioconversion step (b). According to these embodiments, the stream (F1) may feed several reactors or fermentors operated batchwise in parallel.

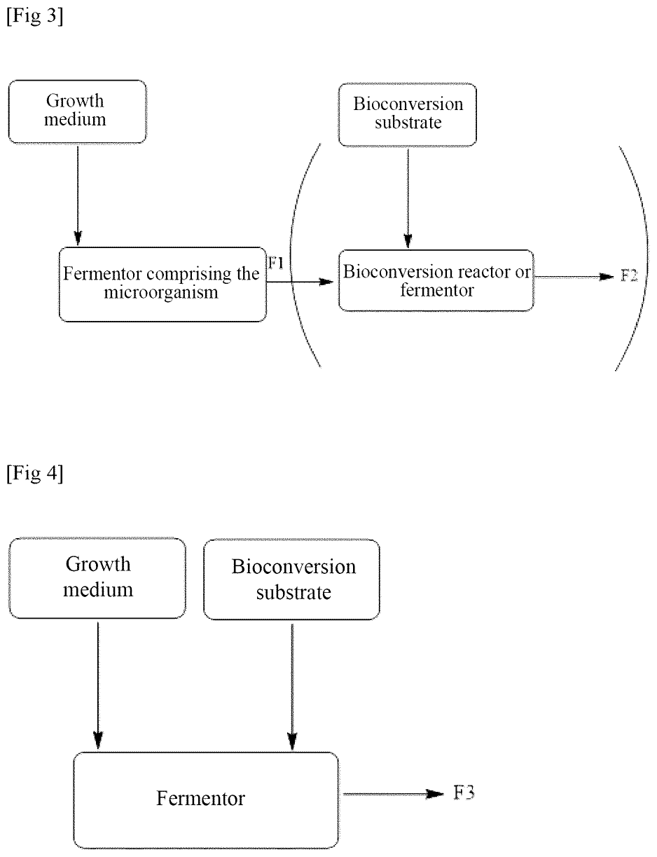

According to another aspect of the present invention, step (b) is carried out in a continuous process at the end of step (a), according to FIG. 3. According to this embodiment, step (a) is carried out in a fermentor. A stream (F1) is drawn from this fermentor to feed a reactor, a fermentor or a reactor or fermentor cascade in which the bioconversion step (b) is carried out. Ferulic acid is fed continuously to the reactor or fermentor in which the bioconversion is carried out. According to this embodiment, the stream (F1) may feed several reactors or fermentors operated continuously in parallel.

According to another aspect of the present invention, step (b) is carried out in a continuous process simultaneously with step (a), according to FIG. 4. According to this embodiment, the fermentor is continuously fed with growth medium and fermentation substrate. A stream (F3) may be drawn comprising compound of formula (I), preferably vanillin, and optionally fermentation substrate, impurities and biomass.

The continuous processes for growing and producing the compound of formula (I), preferably vanillin, according to the present invention, are particularly advantageous in that they allow a gain to be made in terms of time and productivity. These processes also allow a gain to be made in terms of reducing the frequency of maintenance operations. The risk of contamination in the reactor or fermentor, which is operated continuously, is also reduced, since the biomass concentration is high compared to the concentration of possible contaminant; the risk of colonization by a contaminant is thus reduced. Finally, the composition of the biomass is more homogeneous, unlike batchwise processes wherein the biomass composition could vary from one growth batch to another. This homogeneity makes it possible to obtain a more homogeneous crude vanillin composition. This makes it possible to facilitate, in particular, the purification processes.

In the context of the present invention, step (b) may be followed by a step (c) of purifying the compound of formula (I), preferably vanillin, obtained. In general, the purification comprises a first step wherein the fermentation must is separated from the biomass. The purification of the compound of formula (I), preferably of the vanillin, obtained at the end of step (b) may in particular be carried out by liquid/liquid extraction, by distillation, by crystallization, by evaporation in a scraped film evaporator, a falling film evaporator, in a partition evaporator, and/or by stripping. Nanofiltration and ultrafiltration steps may also be carried out as part of the purification step (c). By way of illustration, the purification process may be carried out according to the processes described in WO2013/087795, WO2014/114590, WO2018/146210, WO2021/019005.

According to another embodiment, the vanillin may be extracted continuously from the fermentor by means of a vanillin separation device. By way of illustration, the purification process may be carried out according to the process described in WO2017/025339. There is no particular limitation regarding the choice of the vanillin separation device. Preferably, the vanillin separation device is a membrane filtration device. This type of device makes it possible to recover, from fermentation must, a retentate containing the microorganism and a filtrate free of microorganism. The vanillin separation device may also allow the selective extraction of the vanillin from the extracted fermentation must.

The present invention also relates to a compound of formula (I), preferably a vanillin, which may be obtained according to the process of the present invention. Finally, the present invention refers to the use of the compound of formula (I), preferably vanillin, obtained according to the present invention, as a flavor in the field of human and animal food, or pharmacy, or as a fragrance in the cosmetics industry, perfumery and detergency.

### EXAMPLES

### Example 1: Culturing

The Streptomyces setonii ATCC 39116 strain is cultured under the conditions described in document WO2017/025339.

### Example 2: Continuous Process According to the Invention

The microorganism Streptomyces setonii is cultured according to example 1.

When the glucose concentration in the medium drops below 3 g/l, a new growth medium containing glucose at 30 g/l and MgSO₄·7H₂O at 0.8 g/l is introduced continuously into the fermentor. This medium was previously sterilized. A drain line withdraws the growth medium at the same flow rate as the inflow rate of the growth medium so as to keep the fermentor volume constant. The culture conditions are identical to those of example 1. Under these conditions, the biomass concentration remains constant and the culture is maintained for 13 days.

No degradation of biomass is observed during the 13-day culture period.

### Example 3: Continuous Process According to the Invention

The microorganism Streptomyces setonii is cultured according to example 1.

When the glucose concentration in the medium drops below 3 g/l, a new growth medium containing glucose at 30 g/l and MgSO₄·7H₂O at 0.8 g/l is introduced continuously into the fermentor. This medium was previously sterilized. A drain line withdraws the growth medium at the same flow rate as the inflow rate of the growth medium so as to keep the fermentor volume constant. The culture conditions are identical to those of example 1. After 146 h of growth (about 6 days), a volume of growth medium containing the biomass is withdrawn from the fermentor. The same volume of growth medium containing glucose at 30 g/l and MgSO₄·7H₂O at 0.8 g/l is introduced instantaneously into the fermentor. The feeding and draining are then stopped. The aeration, temperature and stirring conditions do not change. After 8.5 h, the glucose concentration is less than 4 g/l. The feeding with growth medium containing glucose at 30 g/l and MgSO₄·7H₂O at 0.8 g/l is restarted, as is the withdrawal via the drain line.

Biomass degradation is not observed during the sudden variation in growth medium composition. It is also noted that when growth medium feeding and withdrawal are restarted continuously, the growth of the microorganism continues without change.

### Example 4: Continuous Process According to the Invention

The microorganism Streptomyces setonii is cultured according to example 1.

When the glucose concentration in the medium drops below 3 g/l, a new growth medium containing glucose at 30 g/l and MgSO₄·7H₂O at 0.8 g/l is introduced continuously into the fermentor. This medium was previously sterilized. A drain line withdraws the growth medium at the same flow rate as the inflow rate of the growth medium so as to keep the fermentor volume constant. The culture conditions are identical to those of example 1.

After 146 h of growth (about 6 days), a volume of growth medium containing the biomass is withdrawn from the fermentor. The same volume of growth medium containing glucose at 30 g/l and MgSO₄·7H₂O at 0.8 g/l is introduced instantaneously into the fermentor. The feeding and draining are then stopped. The aeration, temperature and stirring conditions do not change. After a further period of 146 h of growth, a volume of growth medium containing the biomass is withdrawn from the fermentor. The same volume of growth medium containing glucose at 30 g/l and MgSO₄·7H₂O at 0.8 g/l is introduced instantaneously into the fermentor. The feeding and draining are then stopped. The aeration, temperature and stirring conditions do not change. Biomass degradation is not observed during sudden variations in growth medium composition. It is also noted that with each of these variations, the microorganism is able to continue its growth.

### Example 5: Vanillin Preparation

Biomass-containing growth medium prepared according to examples 2, 3 or 4 is used. The pH of the growth medium is adjusted to 8.4 by adding 30 mol % sodium hydroxide and kept constant, with stirring.

A solution containing ferulic acid (78 g), 30 mol % sodium hydroxide (60 g), and water (522 g) is prepared. This solution is then added to the fermentor containing the biomass.

Whatever the method of preparing the biomass, a vanillin concentration of about 15 g/l, and 0.3 g/l ferulic acid are measured after 24 hours by HPLC. The ferulic acid conversion rate is about 99%. The vanillin selectivity of the process is greater than 80%.

### Example 6: Preparation of Vanillin According to FIG. 3

The microorganism Streptomyces setonii is cultured in a fermentor 1 according to example 1.

When the glucose concentration in the medium drops below 3 g/l, a new growth medium containing glucose at 30 g/l and MgSO₄·7H₂O at 0.8 g/l is introduced continuously into the fermentor 1 at a flow rate F1. This medium was previously sterilized. A drain line withdraws the growth medium at the same flow rate F1 as the inflow rate of the growth medium so as to keep the volume of the fermentor 1 constant. The culture conditions are identical to those of example 1.

This stream F1 feeds a fermentor 2 also fed with a stream of a 20 g/l ferulic acid solution at a flow rate F2. A drain line withdraws the bioconversion medium at a flow rate F3 which is the sum of the flow rate F1 and the flow rate F2 so as to keep the volume of the fermentor 2 constant. The bioconversion conditions are identical to those of example 5.

The stream drawn off at a flow rate F3 from the fermentor 2 is analyzed at regular intervals. A vanillin concentration of about 5 g/l, and 0.5 g/l of ferulic acid are measured by HPLC. The results obtained show a ferulic acid conversion rate of about 95%. The vanillin selectivity of the process is greater than 80%.

Thus, examples 5 and 6 show that the process for continuously growing the microorganism has no detrimental effect on the microorganism. The microorganism allows the bioconversion of a substrate into vanillin as efficiently as the processes of the prior art wherein the growth is carried out batchwise.

However, the continuous growth processes as described in examples 2 to 4 allow a gain in terms of time and productivity of about 20%. These processes also allow a gain in terms of reducing the frequency of maintenance and cleaning operations. The sizing of the equipment required for the production of an annual volume of vanillin is reduced.

1. A continuous process comprising:
a step (a) of growing a microorganism capable of converting a substrate into a compound of formula (I), wherein the compound of formula (I) is 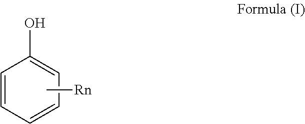
wherein n is between 0 and 5,
R, which may be identical or different, is selected from the group consisting of OH, OR₁, formyl —CHO, —CO₂H, optionally substituted linear or branched alkyl chains comprising between 1 and 6 carbon atoms, optionally substituted linear or branched alkenyl chains comprising between 1 and 6 carbon atoms, and combinations thereof, wherein R₁ is selected from the group consisting of methyl, ethyl, propyl and butyl.

2. The continuous process as claimed in claim 1, wherein the growing the microorganism is carried out in a growth medium.

3. The continuous process as claimed in claim 2, wherein the growth medium is continuously added.

4. The continuous process as claimed in claim 1, wherein a concentration of a carbon source in a growth medium is controlled and is adjusted to the needs of the microorganism capable of converting a substrate into a compound of formula (I).

5. The continuous process as claimed in claim 1, wherein step (a) is preceded by a step (a0) of culturing the microorganism.

6. The continuous process as claimed in claim 1, wherein a substrate is selected from the group consisting of glucose, ferulic acid, eugenol, isoeugenol, coumaric acid, caffeic acid, L-tyrosine, vanillic acid, 4-vinyl guaiacol, and combinations thereof.

7. A process for producing a compound of formula (I), the process comprising:
a step (a) of continuously growing a microorganism capable of converting a substrate into compound of formula (I) as defined in claim 1.

8. The process for producing the compound of formula (I) as claimed in claim 7, further comprising a bioconversion step (b).

9. The process for producing the compound of formula (I) as claimed in claim 8, wherein steps (a) and (b) are successive.

10. The process for producing the compound of formula (I) as claimed in claim 8, wherein steps (a) and (b) are simultaneous.

11. The continuous process for producing the compound of formula (I) as claimed in claim 1, wherein the compound of formula (I) is vanillin.

12. The continuous process for producing the compound of formula (I) as claimed in claim 1, wherein a substrate is selected from the group consisting of glucose, ferulic acid, eugenol, and combinations thereof.

13. The continuous process for preparing the compound of formula (I) as claimed in claim 1, wherein n is between 1 and 2.

14. The process for producing the compound of formula (I) as claimed in claim 7, wherein the compound of formula (I) is vanillin.
